(19) United States
(12) Patent Application Publication
(10) Pub. No.: US 2017/0209133 A1
(43) Pub. Date: Jul. 27, 2017

(54) MEDICAL DEVICES AND RELATED METHODS OF USE

(71) Applicant: Boston Scientific Scimed, Inc., Maple Grove, MN (US)

(72) Inventors: Ronald CIULLA, Westford, MA (US); Timothy HARRAH, Cambridge, MA (US); Aaron KIRKEMO, Gladstone, NJ (US); Brandon CRAFT, Edgewater, MD (US); Elizabeth STOKLEY, Baltimore, MD (US); Sebastian KOERNER, Berlin (DE); Anant SUBRAMANIAM, Owings Mills, MD (US)

(73) Assignee: Boston Scientific Scimed, Inc., Maple Grove, MN (US)

(21) Appl. No.: 15/410,166

(22) Filed: Jan. 19, 2017

Related U.S. Application Data
(60) Provisional application No. 62/281,449, filed on Jan. 21, 2016.

(51) Int. Cl. A61B 17/02

(52) U.S. Cl. CPC A61B 17/0218; A61B 2017/0225; A61B 2217/007; A61B 2017/22038

(57) ABSTRACT

A medical device may include a radially expandable sheath extending between a proximal end and a distal end and defining a lumen therein. The sheath may be expandable between a first configuration and a second configuration. The medical device may further include a plurality of expansion support members positioned along the sheath. Each of the expansion support member may extend circumferentially about a longitudinal axis of the sheath.

### CROSS-REFERENCE TO RELATED APPLICATIONS

This patent application claims the benefit of priority under 35 U.S.C. §119 to U.S. Provisional Patent Application No. 62/281,449, filed Jan. 21, 2016, which is herein incorporated by reference in its entirety.

### TECHNICAL FIELD

Examples of the present disclosure relate generally to medical devices and procedures. In particular, embodiments of the present disclosure relate to medical devices and methods for extraction of tissue and/or other materials.

### BACKGROUND

Medical retrieval devices including, for example, baskets and forceps, are often utilized for removing organic material (e.g., blood clots, tissue, and biological concretions such as urinary, biliary, and pancreatic stones) and inorganic material (e.g., components of a medical device or other foreign matter), which may obstruct or otherwise be present within a patient's body cavities. For example, concretions can develop in certain parts of the body, such as in the kidneys, pancreas, ureter, and gallbladder. Minimally invasive medical procedures are used to remove these concretions through natural orifices, or through an incision, such as during a percutaneous nephrolithotomy (PNCL) procedure.

One problem commonly associated with retrieval of such concretions occurs where the stone or other material is too large to be removed through a sheath (e.g., a renal sheath) and/or a scope, e.g., ureteroscope, en bloc (e.g., whole and/or in one piece). For example, the stone or other material may be too large to pass through a lumen defined by the sheath and/or the working channel of such a ureteroscope. In such cases, a medical professional may be required to break up such stones and/or other material to be small enough to pass through the sheath and/or the working channel of such a ureteroscope. Such a process, however, may be lengthy and expensive. Additionally, breaking up such stones or other materials increases the risk of a fragment being left behind by the medical professional during such a procedure, which may pose dangerous health risks for the patient. Accordingly, the medical professional may be required to deliver increasingly larger (e.g., diameter) sheaths or scopes into the patient so as to enable en bloc removal of such stones or other material. However, larger sheaths or scopes may increase patient discomfort and/or injury. Accordingly, it may be preferred to minimize the outer diameter of the scope or sheath.

Additionally, various patient anatomy's may require longer or shorter scopes or sheaths for removal of a stone or other material. While trial and error selection of an appropriately length sheath or scope may be performed, such a process may be tedious and time consuming. Additionally, any such scopes or sheaths may have a tendency to back out or retract proximally of a patient. As such, a medical professional may be required to periodically reposition the scope or sheath during a procedure. Similarly, the placement of one or more guidewires or tools may be required prior to insertion of the scope or sheath. Such guidewires or tools may become dislodged during a procedure and may require further attention from a medical professional to reposition and/or adjust. Such constant repositioning, resizing, and manipulation of sheath, scope, guidewires, and/or tools may be time consuming and expensive.

### SUMMARY

Examples of the present disclosure relate to, among other things, sheaths and methods of using the same. Each of the examples disclosed herein may include one or more of the features described in connection with any of the other disclosed embodiments.

In one example, a medical device may include a radially expandable sheath extending between a proximal end and a distal end and defining a lumen therein. The sheath may be expandable between a first configuration and a second configuration. The medical device may further include a plurality of expansion support members positioned along the sheath. Each of the expansion support member may extend circumferentially about a longitudinal axis of the sheath.

Aspects of the medical device may additionally and/or alternatively include one or more other features. The sheath may include a discontinuous circumference. The sheath may include a continuous circumference. The plurality of expansion support members may be spaced equidistantly along the sheath. The plurality of expansion support members may be spaced irregularly along the sheath. In the first configuration, a radius extending between a central longitudinal axis of the sheath to an inner surface of the sheath may be a first radius, and in the second configuration, a radius extending between a central longitudinal axis of the sheath to an inner surface of the sheath may be a second radius. The second radius may be larger than the first radius. The second radius may be between about 5% and 30% larger than the first radius. The sheath may be passively expandable. The sheath may be actively expandable. In the first configuration, a first circumferential portion of the sheath and a second circumferential portion of the sheath may overlap. In the second configuration, the first circumferential portion of the sheath and the second circumferential portion of the sheath may not overlap. The plurality of expansion support members may include between about 1 and about 20 expansion support members. Each of the plurality of expansion support members may include metal. Each of the plurality of expansion support members may include a material having a first rigidity greater than a rigidity of a remainder of the sheath.

In another example, a medical device may include a radially expandable sheath extending between a proximal end and a distal end and defining a lumen therein. The sheath may be expandable between a first configuration and a second configuration. The medical device may also include a plurality of expansion support members positioned along the sheath. Each expansion support member may extend longitudinally along the sheath between the proximal end and the distal end.

Aspects of the medical device may additionally and/or alternatively include one or more other features. The sheath may be passively expandable. In the first configuration, a radius extending between a central longitudinal axis of the sheath to an inner surface of the sheath may be a first radius, and in the second configuration, a radius extending between a central longitudinal axis of the sheath to an inner surface of the sheath may be a second radius.

In another example, a medical device may include a radially expandable sheath having a discontinuous circumference and extending between a proximal end and a distal end and defining a lumen therein. The sheath may be passively expandable between a first configuration and a second configuration. The medical device may further include a plurality of expansion support members positioned along the sheath. Each expansion support member may extend circumferentially about a longitudinal axis of the sheath. Each of the plurality of expansion support members may include a material having a first rigidity greater than a rigidity of a remainder of the sheath.

Aspects of the medical device may additionally and/or alternatively include one or more other features. In the first configuration, a radius extending between a central longitudinal axis of the sheath to an inner surface of the sheath may be a first radius, and in the second configuration, a radius extending between a central longitudinal axis of the sheath to an inner surface of the sheath may be a second radius.

It may be understood that both the foregoing general description and the following detailed description are exemplary and explanatory only and are not restrictive of the disclosure, as claimed.

As used herein, the terms “comprises,” “comprising,” or other variations thereof, are intended to cover a non-exclusive inclusion such that a process, method, article, or apparatus that comprises a list of elements does not include only those elements, but may include other elements not expressly listed or inherent to such a process, method, article, or apparatus. Additionally, the term “exemplary” as used herein is used in the sense of “example,” rather than “ideal.”

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 illustrates a perspective view of an exemplary sheath including an irrigation channel according to aspects of the disclosure;

FIG. 2 is a cross-sectional view of the sheath of FIG. 1 and further including a sleeve according to aspects of the disclosure;

FIGS. 3A and 3B illustrate an exemplary expandable sheath of the present disclosure in a first configuration;

FIG. 3C illustrates the exemplary expandable sheath of FIGS. 3A and 3B in a second expanded configuration;

FIGS. 4A and 4B illustrate a further exemplary expandable sheath of the present disclosure in a first configuration;

FIG. 4C illustrates the exemplary expandable sheath of FIGS. 4A and 4B in a second expanded configuration;

FIGS. 5A and 5B illustrates a cross-sectional view of a still further exemplary expandable sheath according to aspects of the present disclosure;

FIGS. 6A and 6B illustrate another exemplary expandable sheath of the present disclosure in a first configuration;

FIG. 6C illustrates the exemplary expandable sheath of FIGS. 6A and 6B in a second expanded configuration;

FIG. 7 illustrates an exemplary sheath according to a further aspect of the present disclosure;

FIGS. 8-10 illustrate various views of an exemplary sheath according to another exemplary aspect of the present disclosure;

FIG. 11 illustrates an exemplary sheath according to a further aspect of the present disclosure; and

FIG. 12 illustrates an exemplary sheath according to a still further aspect of the present disclosure.

### DETAILED DESCRIPTION

The terms “proximal” and “distal” are used herein to refer to the relative positions of the components of exemplary medical devices. When used herein, “proximal” refers to a position relatively closer to a user of a medical device. In contrast, “distal” refers to a position relatively farther away from the user of a medical device.

FIG. 1 illustrates an exemplary sheath 10 for insertion in a body of a patient. For example, sheath 10 may include any device configured to allow a user to access internal areas of a subject's body. Additionally or alternatively, sheath 10 may include any device configured to deliver an insertion device (not shown) or other tool therethrough, as will be described in further detail below. That is, sheath 10 may define a lumen 12 configured (e.g., sized and/or shaped) to receive the insertion device, such as, for example, any device configured to allow a user to perform medical diagnoses and/or treatments on a subject, therethrough. According to aspects of the present disclosure, such an insertion device may be a ureteroscope, an endoscope, a hysteroscope, a uteroscope, a colonoscope, a bronchoscope, a cystoscope, a nephroscope, and/or similar devices. Accordingly, an internal diameter of sheath 10 may be between about 1-15 mm, between about 4-10 mm, or about 6-10 mm. As used herein, the terms “about,” “substantially,” and “approximately,” may indicate a range of values within +/−5% of a stated value.

Further, sheath 10 may have any appropriate cross-sectional shape. For example, as shown in FIG. 1, sheath 10 may have a circular cross-sectional shape. However, other cross-sectional shapes, such as ovular, irregular, and/or polygonal cross-sectional shapes are contemplated and are within the scope of this disclosure. Additionally or alternatively, in some examples, the cross-sectional shape and/or size of sheath 10 may vary along the length of sheath 10. For example, in some examples, a proximal portion may have an ovular cross-sectional shape while a distal portion may have a circular cross-sectional shape. Further, in some examples, the diameter of lumen 12 may be varied along the length of sheath 10. Additionally, sheath 10 may have a length of between about 5 and about 30 cm, between about 14 and about 25 cm, or between about 15 and about 20 cm.

Sheath 10 may include any appropriate biocompatible material, such as, for example, polymer, urethane, polyethylene terephthalate (PET), polyvinyl chloride (PVC), acrylic, fluorine-polymer blends, metals (e.g., stainless steel) and the like. In one example, sheath 10 may have a flexibility that is sufficient to allow passage of sheath 10 through a bodily tract or opening (e.g., natural opening(s) or opening(s) made by a medical professional) in a patient's body. For example, sheath 10 may have a flexibility that is sufficient to allow passage of sheath 10 through the urethra and across the bladder of the patient. In such an arrangement, sheath 10 may be a renal sheath. Sheath 10 may be sterile, single-use, and disposable. In other arrangements, however, sheath 10 may be a multiple-use sheath 10 and may be non-disposable.

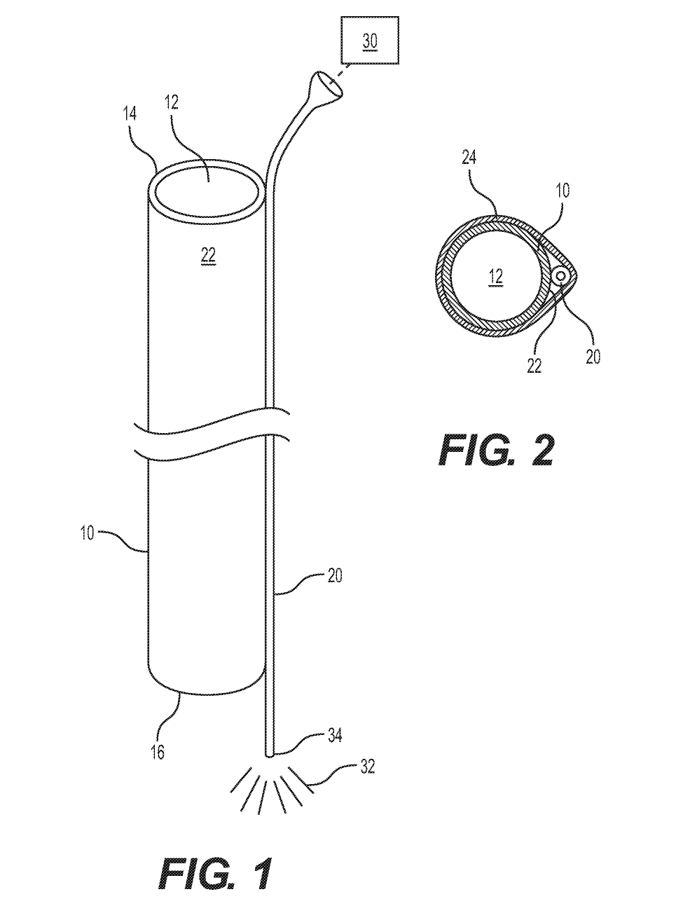

As shown in FIG. 1, sheath 10 may extend between a proximal end 14 and a distal end 16. Further, sheath 10 may include an irrigation channel 20 extending between proximal end 14 and distal end 16. Irrigation channel 20 may be positioned along an external circumferential wall 22 of sheath 10. For example, in some arrangements, irrigation channel 20 may be co-extruded (e.g., monolithically formed as a single-piece structure) with sheath 10. In other arrangements, irrigation channel 20 may be a discrete component adhesively coupled to external circumferential wall 22, and/or heat applied to external circumferential wall 22. Regardless of the manner of coupling of irrigation channel 20 to sheath 10, a wrap, casing, or other such sleeve 24 (FIG. 2) may be positioned so as to receive both sheath 10 and irrigation channel 20 therein. That is, sleeve 24 may encase, encircle, and/or surround on all sides both of sheath 10 and irrigation channel 20. For example, as shown in the cross-sectional view of FIG. 2, sleeve 24 may be tightly (e.g., snug, close) fit about sheath 10 and irrigation tube 20 so as to minimize a size (e.g., profile, diameter) of the combined sheath 10 and irrigation tube 20. That is, an internal surface of sleeve 24 may contact external circumferential surface 22 and an exterior surface of irrigation channel 20. Additionally, it is understood that sleeve 24 may extend the entire length of sheath 10, between proximal end 14 and distal end 16. Alternatively, sleeve 24 may span a length less than the entire length of sleeve 10. That is, sleeve 24 may extend less than the entire length between proximal end 14 and distal end 16 of sheath 10. In addition, sleeve 24 may be non-continuous. That is, sleeve 24 may have one or more sleeve portions (not shown) spaced (e.g., equidistantly or irregularly) along a longitudinal axis of sleeve 10.

Further, it is understood that any one or more of an internal wall of sheath 10 defining lumen 12, external circumferential wall 22, irrigation channel 20, and sleeve 24 may be comprised or be coated with a suitable friction reducing material such as TEFLON®, polyetheretherketone, polyimide, nylon, polyethylene, or other lubricious polymers, to reduce surface friction with the surrounding tissues and/or an insertion device or tools delivered through lumen 10.

Irrigation channel 20, as shown in FIG. 1, may extend proximally of proximal end 14 of sheath 10 so as to couple to a source 30 of sterile irrigation fluid 32. Accordingly, source 30 may contain one or more of water, saline, medicaments, and cleaning solution. The medical professional may, during the course of a procedure, determine a need for irrigation fluid 32 to be delivered through irrigation channel 20 to either clear an area of debris, clean or remove biological matter (e.g., blood) from an insertion device or tool delivered through lumen 12 of sheath 10, or to facilitate healing. Accordingly, the medical professional may selectively actuate an actuator (not shown) to enable source 30 to deliver irrigation fluid 32 through irrigation channel 20 and distally thereof, as shown in FIG. 1. For example, actuation of the actuator may open a valve within or proximal of irrigation channel 20 such that irrigation fluid 32 may be delivered from source 30 to irrigation channel 20. It is understood that any appropriate valve and actuator, as is known in the art, may be employed to fluidly couple source 30 and irrigation channel 20. Further, it is understood that a pressurizer (e.g., pump) or the like may be coupled to source 30, and/or fluidly coupled to irrigation channel 20, so as to deliver irrigation fluid 32 from source 30 to irrigation channel 20 and distally thereof under pressure. Further, as shown in FIG. 1, a distal end 34 of irrigation channel 20 may extend distally of distal end 16 of sheath 10. Accordingly, irrigation channel 20 may deliver irrigation fluid from source 30 to a location downstream (e.g., distal) of sheath 20 so as to clear debris or a field of view distal of distal end 16.

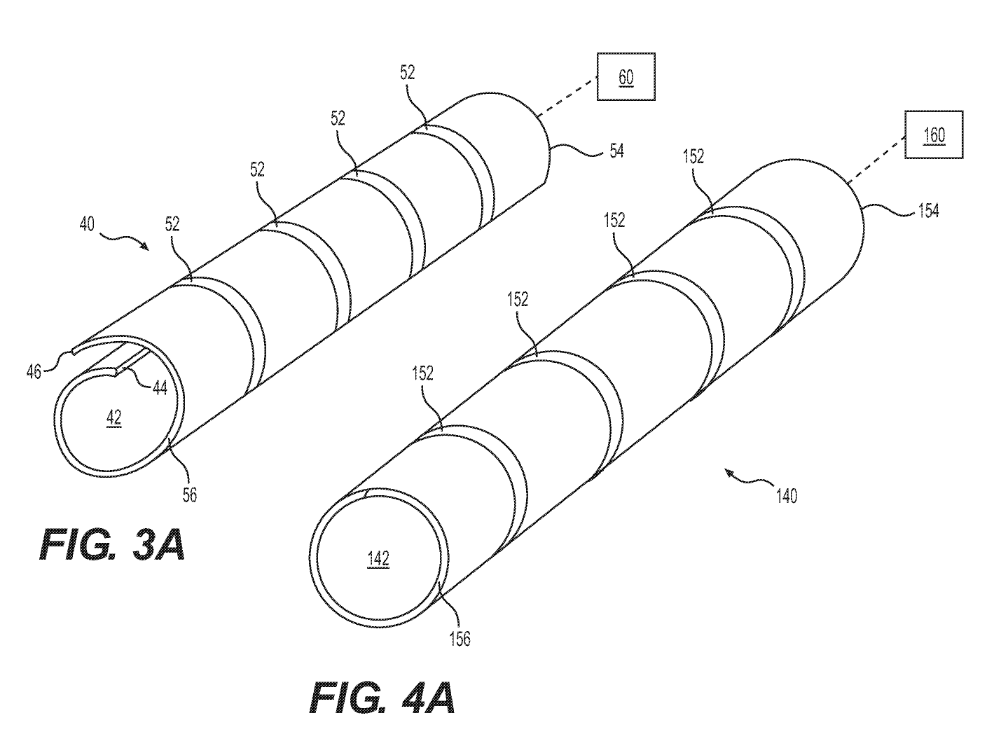

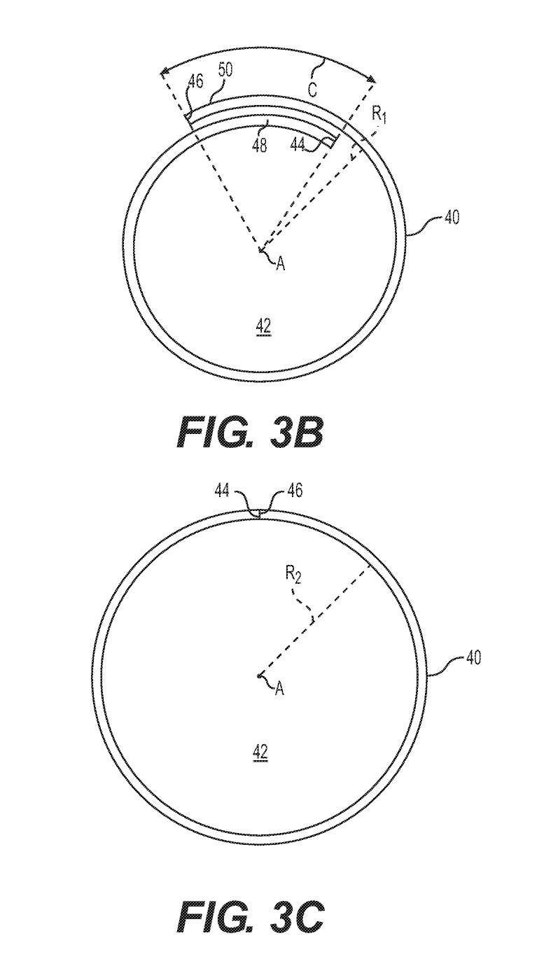

FIGS. 3A and 3B illustrate an exemplary sheath 40 of the present disclosure in a first configuration. Sheath 40 may be similar to sheath 10, and as such, may include any device configured to allow a user to access internal areas of a subject's body and/or any device configured to deliver an insertion device (not shown) or other tool therethrough. Further, sheath 40 may be similarly dimensioned, shaped, and/or comprised of similar materials as sheath 10, as described above. Sheath 40, however, may define a selectively, radially expandable lumen 42 therethrough. For example, in a first configuration, as shown in FIG. 3B, lumen 42 of sheath 40 may have a radius R₁ extending from a central longitudinal axis A of sheath 40 toward an inner surface of an outermost portion of sheath 40. In a second expanded configuration, as shown in FIG. 3C, however, lumen 42 of sheath 40 may have a radius R₂ extending from the central longitudinal axis A of sheath 40 toward an inner surface of an outermost portion of sheath 40. As shown in FIGS. 3B and 3C, radius R₂ is larger than radius R₁. For example, radius R₂ may be between about 5% and about 30%, between about 10% and about 25%, or between about 12% and 18% larger than radius R₁. Alternatively, radius R₂ may be between about 5% and about 300%, between about 60% and about 250%, or between about 100% and 200% larger than radius R₁. That is, in the second expanded configuration, as shown in FIG. 3C, lumen 42 of sheath 40 may define a cross-sectional area larger than in the first configuration, as shown in FIG. 3B. Accordingly, in the second expanded configuration, en bloc removal of stones or other material having a dimension exceeding the cross-sectional area of lumen 42 in the first configuration may be achieved. That is, a single sheath 40 having a selectively expandable radial dimension may enable stones or other material having varying sizes and shapes to be removed through lumen 42 of sheath 40 without necessitating removal of sheath 40 and insertion of an additional larger sheath.

With continuing reference to FIGS. 3B and 3C, sheath 40 may include a discontinuous circumference. For example, as shown, sheath 40 may be cut, sliced, or otherwise machined so as to form a roll or wrap. Alternatively, a flat piece of material may be rolled, bent, or otherwise machined to form a roll or wrap. In either arrangement, sheath 40 may include a first end 44 and a second end 46. As shown, sheath 40 may extend from first end 44 circumferentially about central longitudinal axis A (e.g., in the counter-clockwise direction as shown in FIG. 3A) to second end 46 so as to define an overlapping roll, wrap, or spiral. That is, a first portion 48 of sheath 40 circumferentially nearer to first end 44 and a second portion 50 of sheath 40 circumferentially nearer to second end 46 may overlap along an arcuate length C defined about central longitudinal axis A of sheath 40. In some arrangements, for example, radius R₂ may be about 15 French (5 mm) and radius R₁ may be about 9 French (3 mm). In such an example, first portion 48 of sheath 40 circumferentially nearer to first end 44, and a second portion 50 of sheath 40 circumferentially nearer to second end 46 may overlap along an arcuate length C of about 8.72 mm (about 46% of 2×R₁). In another arrangement, for example, radius R₂ may be about 15 French (5 mm) and radius R₁ may be about 12.5 French (4.17 mm). In such an example, first portion 48 of sheath 40 circumferentially nearer to first end 44 and a second portion 50 of sheath 40 circumferentially nearer to second end 46 may overlap along an arcuate length C of about 12.5% of 2×R₁. In another arrangement, sheath 40 may completely overlap (e.g., 100%) such that sheath 40 may double in size. For example, first portion 48 of sheath 40 circumferentially nearer to first end 44 and a second portion 50 of sheath 40 circumferentially nearer to second end 46 may completely overlap one another in the first configuration and expand to the second expanded configuration (FIG. 3B) such that sheath 40 may double in radial dimension.

When expanded, as described in further detail below, first portion 48 and second portion 50 may expand, dilate, spread, or otherwise move apart from one another such that they no longer overlap (FIG. 3C), or, such that they overlap along an arcuate length (not shown) defined about central longitudinal axis A less than the arcuate length C. If, for example, first portion 48 and second portion 50 are moved apart so as to no longer overlap, as shown in FIG. 3C, first end 44 and second end 46 may be positioned immediately next to one another, and/or contact or abut one another. In such a manner, lumen 42 may be selectively configured to pass various sized stones or other material therethrough en bloc.

To facilitate radial expansion of sheath 40, one or more expansion support members 52 may be positioned axially along sheath 40. For example, as shown in FIG. 3A, a plurality of expansion support members 52 may be equidistantly spaced along sheath 40. In other arrangements, however, expansion support members 52 may be irregularly spaced along sheath 40. Additionally, while 4 expansion support members 52 are illustrated in FIG. 3A, such an illustration is merely for explanation purposes. Rather, sheath 40 may include any appropriate number of expansion support members 52 extending between a proximal end 54 and a distal end 56 of sheath 40 may be included. For example, in some arrangements, sheath 40 may include between about 1 and about 20, between about 2 and about 15, or between about 3 and about 10 expansion support members 52. It is understood that the number of expansion support members 52 and the spacing therebetween may be varied and/or selected depending on the length of sheath 40 and/or the intended use or procedure for sheath 40. Further, it is understood that expansion support members 52 may be embedded within the thickness (e.g., between a radially exterior surface of sheath 40 and a radially interior surface of sheath 40 relative to longitudinal axis A). Alternatively, expansion support members 52 may extend along the radially exterior surface of sheath 40, or along the radially interior surface of sheath 40.

Expansion support members 52 may include any structure configured for radial expansion. For example, expansion support members 52 may be comprised of arcuate lengths of spring metal (e.g., leaf springs) or similar constructions extending along the circumferential length of sheath 40 between first end 44 and second end 46. Alternatively, each expansion support member 52 may include two (or more) members coupled together via one or more hinge points (not shown). In some arrangements, expansion support members 52 may be single or multi-layered. For example, expansion support members may include varying polymers along the length of sheath 40 so as to vary radial expansion. In any such arrangement, expansion support members 52 may enable radial expansion of sheath 40 between the first configuration (FIG. 3B) and the second expanded configuration (FIG. 3C). For example, expansion support members 52 may provide structural support and integrity to control the degree of dilation, spreading, or expansion of first end 44 away from second end 46 of sheath 40.

Further, it is understood that expansion support members 52 may be configured for either passive or active expansion. For example, in use, sheath 40 may be inserted into the body of a patient in the first configuration as shown in FIGS. 3A and 3B. That is, sheath 40 may be biased towards the first configuration. Various stones or other materials may be retrieved from within the body of the patient through lumen 42 of sheath 40. Upon the attempted retrieval of a stone or other material having a dimension larger than an internal diameter (e.g., 2 x R₁) of lumen 42 of sheath 40, however, expansion support members 52 may deflect, open, or expand so as to transition toward the second expanded configuration, as shown in FIG. 3C, in which the internal diameter (e.g., 2×R₂) of lumen 42 is sufficiently large so as to receive the stone or other material therethrough. That is, each expansion support member 52 may passively deflect so as to enlarge the internal diameter of lumen 42 to accommodate various sized stones or other materials for retrieval therethrough. It is understood that each expansion support member 52 may passively expand individually of any other expansion support member 52. Further, once such a stone or other material is passed through lumen 42, each expansion support member may return (e.g., radially collapse) and return to the first configuration as shown in FIG. 3B.

Alternatively, as noted above, each expansion support member 52 may be configured to actively expand. In such an arrangement, an actuator 60 (schematically illustrated in FIG. 3A) may be positioned near proximal end 54 of sheath 40, or may associated with a handle (not shown) coupled to proximal end 54 of sheath 40. It is understood that actuator 60 may include, by way of non-limiting example, at least one of a slider, a push button, a rocker, a lever, a wheel, a plunger, or any combination thereof. Actuator 60 may be operably coupled to each expansion support member 52 via any appropriate linkage or other mechanical connection such that actuation of actuator 60 may urge expansion support members 52, and consequently, sheath 40 between the first configuration (FIG. 3B) and the second expanded configuration (FIG. 3C).

In one example, a proximal end of each of first end 44 and second end 46, and/or a first end of each expansion support member 52 adjacent first end 44, and a second end of each expansion support member 52 adjacent second end 46 of sheath 40, may be coupled to a wire (not shown) embedded within or coupled along sheath 40 and extending proximally towards actuator 60. In such an arrangement, actuator 60 may include a cam hub mechanism in which two rotatable or swiveling members (e.g., gears or linkages) of the actuator 60 may be counter rotated (e.g., rotated in opposite directions) so as to open or expand sheath 40. The two rotatable or swiveling members may be manually or automatically rotated or moved via a motor.

In another arrangement, each expansion support member 52 may be comprised of a shape memory alloy (SMA), such as, for example, NITINOL™. Such materials, upon activation (e.g., application of heat and/or exposure to body chemistry), reform to a “remembered” shape. That is, upon application of an activating force from actuator 60, expansion support members 52 may transition to its “remembered” configuration and expand sheath 40. In such an arrangement, each expansion support member 52 may be electrically coupled via a conductor to actuator 60. For example, one or more conductive wires (not shown) may be embedded within or extend along sheath 40 toward actuator 60. Upon actuation of actuator 60 by a medical professional, such conductive wires may deliver current to one or more of the expansion support members 52 to selectively heat, and therefore, expand the expansion support members 52 and thereby, sheath 40.

In yet a further arrangement, each expansion support member may be selectively inflatable. For example, in some arrangements, each expansion support member 52 may include a balloon or other such inflatable member. Additionally, an inflation lumen (not shown) may be embedded within or extend along sheath 40 toward actuator 60 and may be fluidly coupled to each expansion support member 52. Upon actuation of actuator 60 by a medical professional, inflation fluid (e.g., gas, saline, water, etc.) may be delivered through the inflation lumen to one or more expansion support members 52 so as to “inflate” or expand each expansion support member 52, and therefore, sheath 40. In such an arrangement, actuator 60 may comprise a syringe.

In any such arrangement, however, each expansion support member 52 may actively deflect so as to enlarge the internal diameter of lumen 42 to accommodate various sized stones or other materials for retrieval therethrough. Additionally, it is understood that each expansion support member 52 may actively expand individually of any other expansion support member 52. In such arrangements, actuator 60 may include any appropriate number of actuation features so as to individually expand each expansion support member 52.

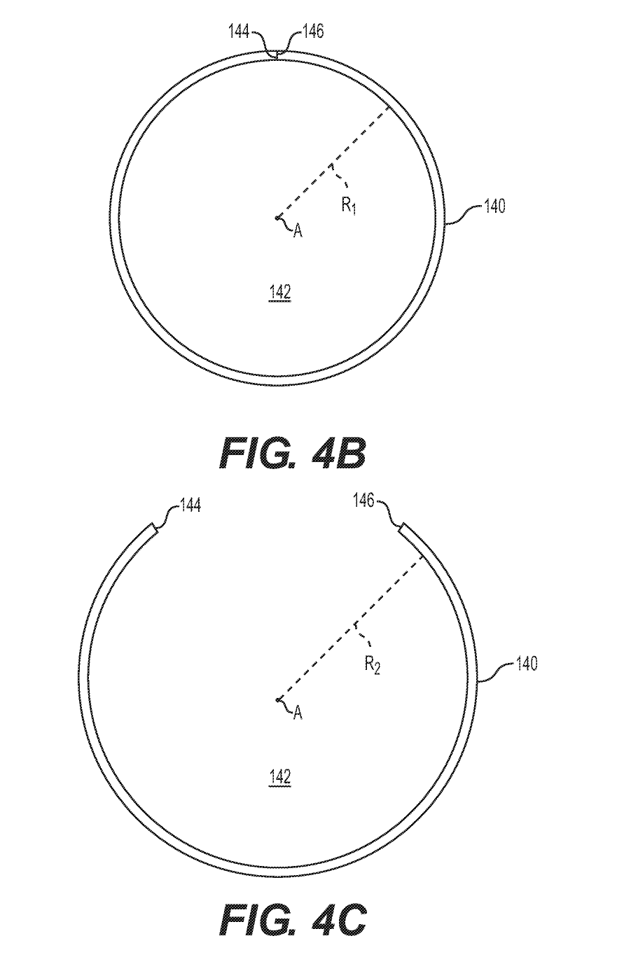

FIGS. 4A-4C illustrate a further exemplary sheath 140 according to the present disclosure. Sheath 140 may be similar in manner of operation and construction as sheath 40 except where described below. Accordingly, components of FIGS. 4A-4C with the same or similar functions as those in FIGS. 3A-3C are shown with the same reference number plus 100. Similar to sheath 40, sheath 140 may extend between a proximal end 154 and a distal end 156, and may define a selectively, radially expandable lumen 142 therethrough. For example, in a first configuration, as shown in FIG. 4B, lumen 142 of sheath 140 may have a radius R₁ extending from a central longitudinal axis A of sheath 140 toward an inner surface of an outermost portion of sheath 140. In a second expanded configuration, as shown in FIG. 4C, however, lumen 142 of sheath 140 may have a radius R₂ extending from the central longitudinal axis A of sheath 140 toward an inner surface of an outermost portion of sheath 140. As shown in FIGS. 4B and 4C, radius R₂ is larger than radius R₁.

Similar to sheath 40, sheath 140 may include a discontinuous circumference. However, rather than a first end 144 and a second end 146 overlapping one another in the first configuration (FIG. 4B), first end 144 and second end 146 may be positioned immediately next to one another, and/or contact or abut one another. When expanded to the second expanded configuration (FIG. 4C), (e.g., via either passive or active expansion of one or more expansion support members 152) first end 144 and a second end 146 may expand, dilate, spread, or otherwise move apart from one another so as to form a c-shaped lumen 142. In such a manner, lumen 142 may be selectively configured to pass various sized stones or other material therethrough en bloc.

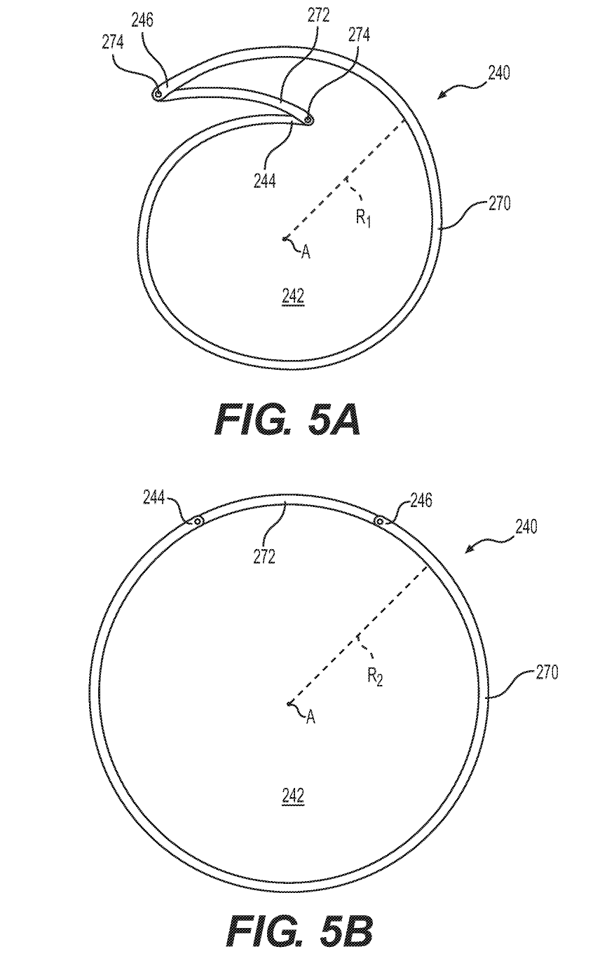

FIGS. 5A and 5B illustrate cross-sectional views of a sheath 240 according to an additional aspect of the present disclosure, in a first configuration and a second expanded configuration, respectively. Sheath 240 may be similar in manner of operation and construction as sheaths 40 and 140 except where described below. Accordingly, components of FIGS. 5A and 5B with the same or similar functions as those in FIGS. 4A-4C are shown with the same reference number plus 100. Similar to sheath 140, sheath 240 may extend between a proximal end (not shown) and a distal end (not shown), and may define a selectively, radially expandable lumen 242 therethrough. For example, in a first configuration, as shown in FIG. 5A, lumen 242 of sheath 240 may have a radius R₁ extending from a central longitudinal axis A of sheath 240 toward an inner surface of an outermost portion of sheath 240. In a second expanded configuration, as shown in FIG. 5B, however, lumen 242 of sheath 240 may have a radius R₂ extending from the central longitudinal axis A of sheath 240 toward an inner surface of an outermost portion of sheath 240. As shown in FIGS. 5A and 5B, radius R₂ is larger than radius R₁.

In contrast to sheaths 40 and 140, however, sheath 240 includes a continuous (e.g., closed) circumference. That is, sheath 240 may include a first portion 270 and a second portion 272. Second portion 272 may be coupled to first portion 270 between first end 244 and second end 246 of first portion 270. In some exemplary arrangements, first portion 270 may be comprised of a material having a first degree of flexibility, and second portion 272 may be comprised of a material having a second degree of flexibility, different than the first degree of flexibility. For example, the second degree of flexibility may be greater than the first degree of flexibility. In some arrangements, second portion 272 may include a different material and/or geometric properties or dimensions (e.g., thickness) than first portion 270. Further, in some arrangements, second portion 272 may be coupled to first portion 270 via one or more rotatable hinge points 274. Hinge points may reduce the degree of kinking or twisting experienced by sheath 240 when transitioning between the first configuration and the second expanded configuration. That is, hinge points 274 may enable second portion 272 to rotate relative to first portion 270. Accordingly, sheath 240 may move between the first configuration and the second expanded configuration smoothly. When expanded to the second expanded configuration (FIG. 5B), (e.g., via either passive or active expansion of one or more expansion support members (not shown)) sheath 240 may expand, unfurl, dilate, or otherwise open such that second portion 272 moves relative to first portion 270. In doing so, second portion 272 may flex or stretch into the rounded shape of the second expanded configuration, as shown FIG. 5B. In such a manner, lumen 242 may be selectively configured to pass various sized stones or other material therethrough en bloc.

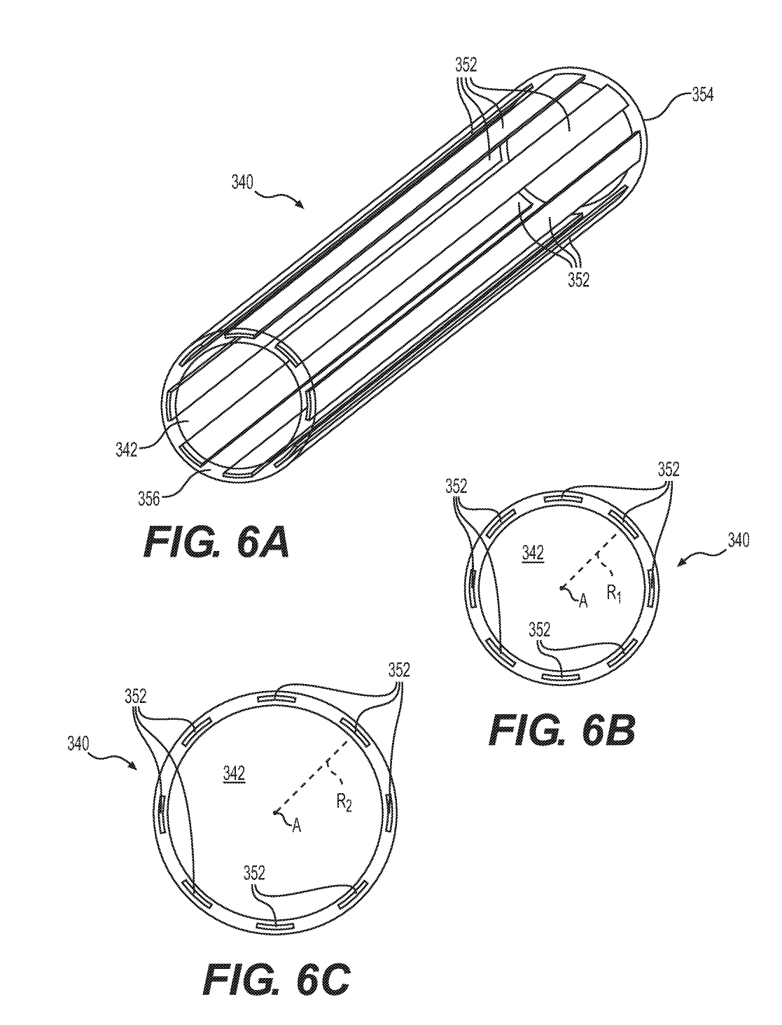

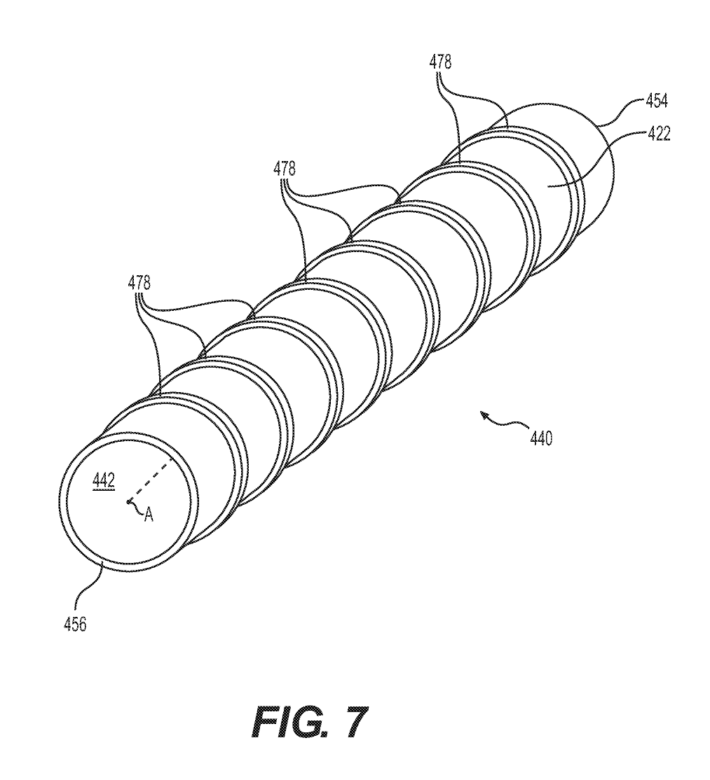

FIGS. 6A-6C illustrate a further exemplary sheath 340 according to the present disclosure. Sheath 340 may be similar in manner of operation and construction as sheath 40, 140, and 240 except where described below. Accordingly, components of FIGS. 6A-6C with the same or similar functions as those in FIGS. 5A and 5C are shown with the same reference number plus 100. Sheath 340 may extend between a proximal end 354 and a distal end 356, and may define a selectively, radially expandable lumen 342 therethrough. For example, in a first configuration, as shown in FIG. 6B, lumen 342 of sheath 340 may have a radius R₁ extending from a central longitudinal axis A of sheath 340 toward an inner surface of an outermost portion of sheath 340. In a second expanded configuration, as shown in FIG. 6C, however, lumen 342 of sheath 340 may have a radius R₂ extending from the central longitudinal axis A of sheath 340 toward an inner surface of an outermost portion of sheath 40. As shown in FIGS. 6B and 6C, radius R₂ is larger than radius R₁.

In contrast to sheaths 40, 140, and 240, however, sheath 340 may include longitudinally extending expansion support members 352, embedded within sheath 340 as shown in FIG. 6 (in which sheath 340 is illustrated as clear or see through to enable a clear view of expansion support members 352). That is, rather than extending about (e.g., radially around) the circumference of sheath 340, expansion support members 352 may extend axially along longitudinal axis A between proximal end 354 and distal end 356. In such an arrangement, expansion support members 352 may include ribs, boning, or the like. Additionally, while 8 expansion support members 352 are illustrated in FIGS. 6A-6C, such an illustration is merely for explanation purposes. Rather, sheath 340 may include any appropriate number of expansion support members 352 extending between a proximal end 354 and a distal end 356 of sheath 340 may be included. For example, in some arrangements, sheath 340 may include between about 1 and about 50, between about 3 and about 40, or between about 5 and about 30 expansion support members 352. Additionally or alternatively, sheath 340 may include as many expansion support members 352 as will fit arranged about the circumference of sheath 340 as possible. In such an arrangement, due to the large number of expansion support members 352, each expansion support member 352 may be tightly packed (e.g., abuts or is minimally spaced) from an adjacent expansion support members 352. It is understood that the number of expansion support members 352 and the spacing therebetween may be varied or selected without departing from the scope of this disclosure.

Further, expansion support members 352 may include a material having a first rigidity while the remainder of sheath 340 is comprised of a material having a second rigidity. In some arrangements, the first rigidity may be greater than the second rigidity. As such, expansion support members 352 may maintain the structural integrity and/or reinforce the remainder of sheath 340. During expansion between the first configuration (FIG. 6B) and the second expanded configuration (FIG. 6C), expansion support members 352 may deflect radially outwardly of central longitudinal axis A of sheath 340 via either passive or active expansion of expansion support members 352. For example, a balloon or similar inflatable member can be delivered through lumen 342 and inflated so as to actively expand sheath 340. When expanded to the second expanded configuration (FIG. 6C), sheath 340 may be configured to pass various sized stones or other material therethrough en bloc.

It is to be understood that in any of the above-disclosed radially expandable sheaths 40, 140, 240, and 340 may be coupled with a locking system (not shown). For example, upon expansion of any of sheaths 40, 140, 240, and/or 340 via either passive or active actuation, a locking system may be actuated to maintain such sheath 40, 140, 240, and/or 340 in the second expanded configuration. For example, each of the expandable sheaths 40, 140, 240, and/or 340 may be comprised of a material that plastically deforms when expanded. Accordingly, upon deflecting radially outward, each of sheaths 40, 140, 240, and/or 340 may be “locked” or remain in the second expanded configuration.

FIG. 7 illustrates an exemplary sheath 440 according to a further aspect of the present disclosure. Sheath 440 may extend between a proximal end 454 and a distal end 456 and define a lumen 442 extending therethrough. Sheath 440 may be similar to sheath 10, 40, 140, 240, and/or 340, and as such, may include any device configured to allow a user to access internal areas of a subject's body and/or any device configured to deliver an insertion device (not shown) or other tool therethrough. Further, sheath 440 may be similarly dimensioned, shaped, and/or comprised of similar materials as sheath 10, as described above.

Sheath 440, however, may additionally include one or more tissue engagement features 478 extending radially outwardly, relative to a central longitudinal axis A of sheath 440, of an external circumferential wall 422 of sheath 440. Tissue engagement features 478 may include a continuous helical spiral thread, a plurality of discrete spiral wraps, or any other such structure extending from the external circumferential wall 422 of sheath 440. Accordingly, upon insertion of sheath 440 into the body of a patient, tissue engagement features 478 may contact tissue in the body of the patient so as to maintain sheath 440 in a selected location within the body of the patient. For example, tissue engagement features 478 may form an interference fit between sheath 440 and tissue within the body of the patient. That is, tissue engagement features 478 may exert a greater radial force on tissue within the body of the patient than remaining portions of sheath 440. As such, tissue will be caused to move between adjacent portions of or between adjacent discrete engagement features 478 and provide resistance so as to prevent sheath 440 from backing out of the body of the patient.

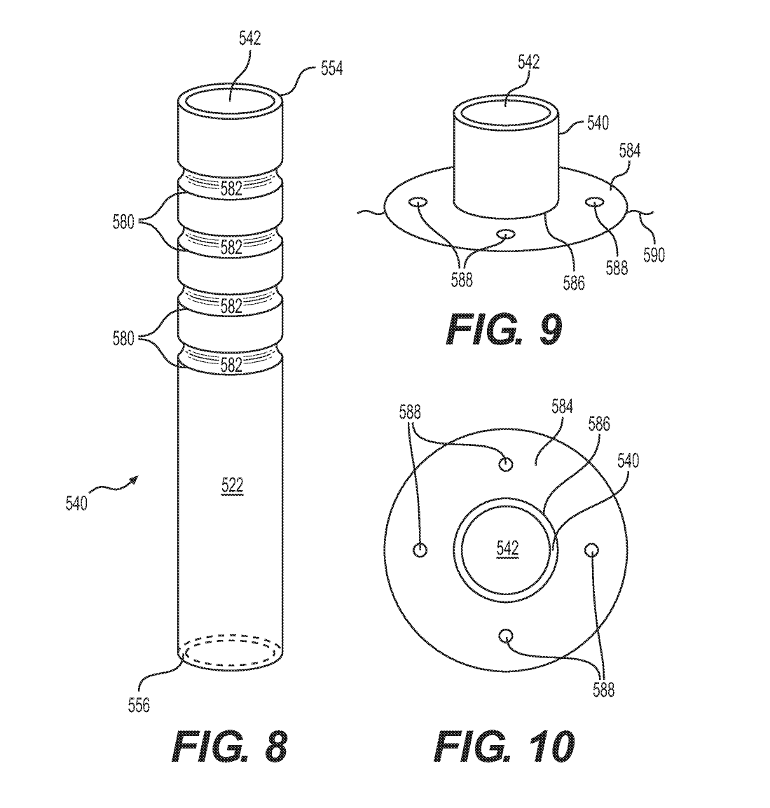

FIGS. 8-10 illustrate various views of an exemplary sheath 540 according to another exemplary aspect of the present disclosure. For example, sheath 540 may extend between a proximal end 554 and a distal end 556 and define a lumen 542 extending therethrough. Sheath 540 may be similar to sheath 10, 40, 140, 240, 340, and/or 440, and as such, may include any device configured to allow a user to access internal areas of a subject's body and/or any device configured to deliver an insertion device (not shown) or other tool therethrough. Further, sheath 540 may be similarly dimensioned, shaped, and/or comprised of similar materials as sheath 10, as described above.

Sheath 540, however, may additionally include one or more discrete grooves, channels, or other such detents 580 positioned adjacent proximal end 554. For example, as shown in FIG. 8, sheath 540 may include a plurality of circumferentially extending detents 580 spaced equidistantly from one another. In other arrangements, however, detents 580 may be irregularly spaced along sheath 540. Additionally, while 4 detents 580 are illustrated in FIG. 8, such an illustration is merely for explanation purposes. Rather, sheath 540 may include any appropriate number of detents 580. For example, in some arrangements, sheath 40 may include between about 1 and about 25, between about 3 and about 15, or between about 3 and about 6 detents 580. It is understood that the number of detents 580 and the spacing therebetween may be varied and/or selected depending on one or more of patient anatomy, the desired length of sheath 540, and the intended use or procedure for sheath 540. Further, in some arrangements, detents may be continuous rather than discrete. For example, in some arrangements, detents 580 may include a continuous helical spiral.

Regardless of the number or arrangement of detents 580, each detent 580 may define a stabilizer reception space 582 having a smaller diameter than a diameter of an external circumferential wall 522 of sheath 540. As such, each detent 580 may be configured (e.g., sized and shaped) to receive a stabilizer 584 (FIGS. 9 and 10) therein. For example, stabilizer 584 may include a disc, washer, ring, plate, or cap include a central opening 586 extending therethrough. Opening 586 may be sized (e.g., include a diameter) sufficient to receive sheath 540 therein, as will be described in further detail below. Additionally, stabilizer may include one or more attachment features 588. Attachment features 588 may include any one or more openings, pockets, and discontinuities through which any one or more sutures, barbs, hooks, or the like (not shown) may be disposed so as to couple stabilizer 584 to skin, fascia, or tissue 590 of the patient. In such a manner, stabilizer 584 may be fixed relative to tissue 590 of the patient during a procedure.

As noted above, opening 586 may be configured to receive sheath 540 therein. For example, sheath 540 may be inserted within opening 586 via a snap-fit or an interference fit between opening 586 and external circumferential surface 522. That is, a diameter of the of external circumferential surface 522 may approximate or be slightly larger than (e.g., between about 0.1 mm and about 2 mm) a diameter of opening 586. Accordingly, when inserted into opening 586, sheath 540 may be radially compressed or tightly held within stabilizer 584. Additionally or alternatively, when inserted into opening 586, stabilizer 584 may be radially expanded. Advancing sheath 540 further may align opening 586 with one of detents 580 defining stabilizer reception space 582. Upon such an alignment, sheath 540 may return to its non-radially compressed or tightly held arrangement within opening 586. Additionally or alternatively, stabilizer 584 may radially collapse to an unexpanded configuration. In either arrangement, upon reception of one of detents 580 within opening 586, sheath 540 may be longitudinally fixed relative to stabilizer 584. Accordingly, sheath 540 may maintain a desired position throughout the course of a procedure. It is understood that one or both of sheath 540 and stabilizer 584 may be comprised of a material having an appropriate balance of rigidity and flexibility to facilitate such an interference fit or snap-fit. Additionally, such materials may enable advancing sheath 540 relative to stabilizer 584 so as to achieve a desired length within the both of a patient. Accordingly, the force exerted by such an interference fit or snap-fit arrangement may be manually overcome by the medical professional. That is, after reception of one of detents 580 within opening 586, sheath 540 may be further advanced and may be radially compressed or tightly held within stabilizer 584 and/or stabilizer 584 may be radially expanded until sheath 540 is advanced to the next (e.g., an adjacent) detent 580 of sheath 540 is axially aligned with opening 586. In such a manner, a medical professional may determine the appropriate length of sheath 540 and may maintain sheath 540 in a fixed position relative to tissue of the patient. In a further example, stabilizer 584 may include a hinge point or flex point (not shown) so as to selectively open and close stabilizer 584 in a manner similar to a clamp. That is, upon opening, sheath 540 may be positioned within opening 586 by moving sheath 540 relative to opening 586 in a radial direction (e.g., a direction substantially perpendicular to a longitudinal axis) of sheath 540.

While sheath 540 has been described as including detents 580 with stabilizer 584 is received within detents 580, the reverse may also be true. That is, in some arrangements, rather than detents 580, sheath 540 may include raised portions (not shown) configured for receipt within a groove, detent, or cavity of stabilizer 584 without departing from the scope of this disclosure. For example, stabilizer 584 may include a negative feature enabling selective coupling and uncoupling from a raised feature positioned along sheath 540.

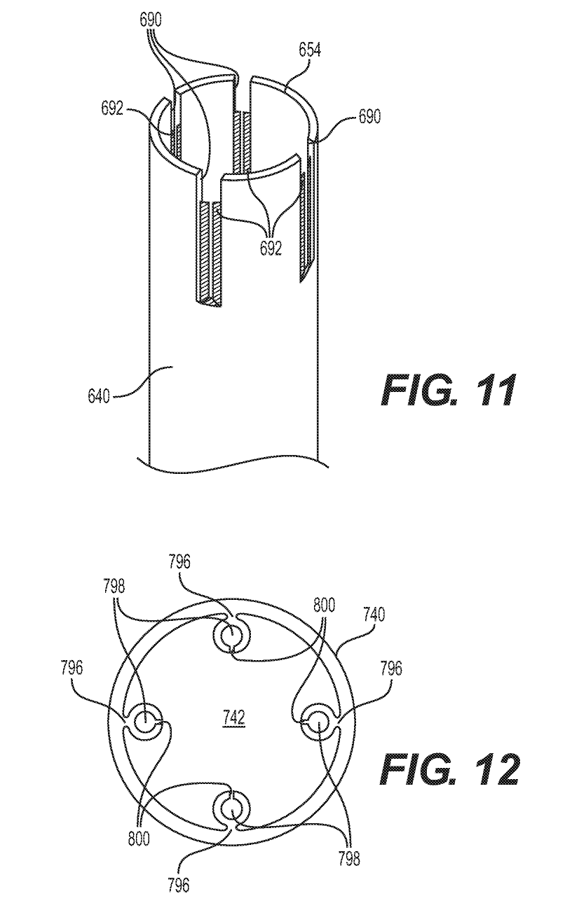

As noted above, sheaths 10, 40, 140, 240, 340, 440, and/or 540 may include any device configured to allow a user to access internal areas of a subject's body and/or any device configured to deliver an insertion device (not shown) or other tool therethrough. In use, one or more such sheaths may be delivered over or along a guide wire or other such device. In use, however, such wires may slip or become dislodged from the sheath. Accordingly, FIGS. 11 and 12 illustrate exemplary mechanisms for maintaining such wires in a desired location along such sheaths.

For example, as shown in FIG. 11, a sheath 640 may include a proximal end 654 defining one or more longitudinally extending slits, openings, or channels 690 therethrough. For example, as shown, sheath 640 may include 4 channels 690 extending distally from proximal end 654 toward a distal end (not shown) of sheath 640. While 4 channels 690 are illustrated in FIG. 11, such an illustration is merely for explanation purposes. Rather, sheath 640 may include any appropriate number of channels 690 and may be selected depending on the number of wires or other such tools that a medical professional may wish to secure. For example, in some arrangements, sheath 640 may include between about 1 and about 10, between about 2 and about 8, or between about 4 and about 6 channels 690. As shown in FIG. 11, each channel 690 may be lined or otherwise filled with a low durometer material 692. By way of example only, material 692 may include any one or more of silicone, rubber, and plastic. Material 692 may be formable, compressible, or otherwise configured to receive a guide wire or other such tool therein. Indeed, upon insertion of such a guide wire or tool within a channel 692, material 692 may grip the guide wire or tool so as to maintain an axial position of the guide wire or tool relative to sheath 640. It is understood that in some arrangements, a separate or discrete cap (not shown) defining one or more channel 690 lined with material 692 may be couplable to proximal end 654 of sheath 640. In such arrangements, the cap may be a separate component that may be selectively coupled or decoupled from the proximal end of sheath 640 via any suitable manner (e.g., snap-fit).

Additionally or alternatively, as shown in FIG. 12, a sheath 740 may define a lumen 742. In addition, one or more retention mechanisms 796 may be included along an inner circumferential wall of sheath 740. For example, as shown in FIG. 12, 4 retention mechanisms 796 may extend from an inner circumferential wall of sheath 740. While 4 retention mechanisms 796 are illustrated in FIG. 12, such an illustration is merely for explanation purposes. Rather, more or less retention mechanisms 796 may extend from an inner circumferential wall of sheath 740 as desired. As shown, each retention mechanisms 796 may include a c-shaped structure defining a lumen 798 therein. Lumen 798 may be configured to receive a guide wire or other such tool therein through opening or slit 800. Accordingly, upon insertion of a guide wire or tool within a channel 798, retention mechanisms 796 may maintain the guide wire or tool in a desired position relative to sheath 740.

While principles of the present disclosure are described herein with reference to illustrative embodiments for particular applications, it should be understood that the disclosure is not limited thereto. Those having ordinary skill in the art and access to the teachings provided herein will recognize additional modifications, applications, embodiments, and substitution of equivalents all fall within the scope of the embodiments described herein. For example, any feature of any of the above described examples may be combined with any other features of the above described examples. By way of example only, any one of sheaths 40, 140, 240, 340, 440, 540, 640, and/or 740 may include irrigation channel 20 extending along an external circumferential surface of said sheath. Additionally or alternatively, any one of sheaths 10, 40, 140, 240, 340, 540, 640, and/or 740 may include one or more tissue engagement features 478 extending radially outwardly, relative to a central longitudinal axis A of said sheath. Additionally or alternatively, any of sheaths 10, 40, 140, 240, 340, 440, 640, and/or 740 may include detents 580 for cooperation with a stabilizer 584. Additionally or alternatively, any of sheaths 10, 40, 140, 240, 340, 440, 540, and/or 740 may include channels 690 lined with material 692. Additionally or alternatively, any of sheaths 10, 40, 140, 240, 340, 440, 540, and/or 640 may include one or more retention mechanisms 796. Accordingly, the disclosure is not to be considered as limited by the foregoing description.

We claim:

1. A medical device, comprising:
a radially expandable sheath extending between a proximal end and a distal end and defining a lumen therein, wherein the sheath is expandable between a first configuration and a second configuration; and
a plurality of expansion support members positioned along the sheath, wherein each expansion support member extends circumferentially about a longitudinal axis of the sheath.

2. The medical device of claim 1, wherein the sheath includes a discontinuous circumference.

3. The medical device of claim 1, wherein the sheath includes a continuous circumference.

4. The medical device of claim 1, wherein the plurality of expansion support members are spaced equidistantly along the sheath.

5. The medical device of claim 1, wherein the plurality of expansion support members are spaced irregularly along the sheath.

6. The medical device of claim 1, wherein, in the first configuration, a radius extending between a central longitudinal axis of the sheath to an inner surface of the sheath is a first radius, and wherein in the second configuration, a radius extending between a central longitudinal axis of the sheath to an inner surface of the sheath is a second radius.

7. The medical device of claim 1, wherein the second radius is larger than the first radius.

8. The medical device of claim 1, wherein the second radius is larger is between about 5% and 30% larger than the first radius.

9. The medical device of claim 1, wherein the sheath is passively expandable.

10. The medical device of claim 1, wherein the sheath is actively expandable.

11. The medical device of claim 1, wherein in the first configuration, a first circumferential portion of the sheath and a second circumferential portion of the sheath overlap.

12. The medical device of claim 11, wherein in the second configuration, the first circumferential portion of the sheath and the second circumferential portion of the sheath do not overlap.

13. The medical device of claim 1, the plurality of expansion support members includes between about 1 and about 20 expansion support members.

14. The medical device of claim 1, wherein each of the plurality of expansion support members includes metal.

15. The medical device of claim 1, wherein each of the plurality of expansion support members includes a material having a first rigidity greater than a rigidity of a remainder of the sheath.

16. A medical device, comprising:
a radially expandable sheath extending between a proximal end and a distal end and defining a lumen therein, wherein the sheath is expandable between a first configuration and a second configuration; and
a plurality of expansion support members positioned along the sheath, wherein each expansion support member extends longitudinally along the sheath between the proximal end and the distal end.

17. The medical device of claim 16, wherein the sheath is passively expandable.

18. The medical device of claim 16, wherein, in the first configuration, a radius extending between a central longitudinal axis of the sheath to an inner surface of the sheath is a first radius, and wherein in the second configuration, a radius extending between a central longitudinal axis of the sheath to an inner surface of the sheath is a second radius.

19. A medical device, comprising:
a radially expandable sheath having a discontinuous circumference and extending between a proximal end and a distal end and defining a lumen therein, wherein the sheath is passively expandable between a first configuration and a second configuration; and
a plurality of expansion support members positioned along the sheath, wherein each expansion support member extends circumferentially about a longitudinal axis of the sheath, wherein each of the plurality of expansion support members includes a material having a first rigidity greater than a rigidity of a remainder of the sheath.

20. The medical device of claim 19, wherein, in the first configuration, a radius extending between a central longitudinal axis of the sheath to an inner surface of the sheath is a first radius, and wherein in the second configuration, a radius extending between a central longitudinal axis of the sheath to an inner surface of the sheath is a second radius.
